(19) United States
(12) Patent Application Publication
(10) Pub. No.: US 2018/0132773 A1
(43) Pub. Date: May 17, 2018

(54) PORTABLE PHLEBOTOMY KIT

(71) Applicant: Orig3n, Inc., Boston, MA (US)

(72) Inventors: Robin Y. Smith, Boston, MA (US); Kate Blanchard, Boston, MA (US)

(21) Appl. No.: 15/808,347

(22) Filed: Nov. 9, 2017

Related U.S. Application Data
(60) Provisional application No. 62/420,731, filed on Nov. 11, 2016.

(51) Int. Cl. A61B 5/15; A61B 5/153; A61B 17/132; A61F 13/00; A61B 50/30; A61F 13/36; A61B 50/31

(52) U.S. Cl. CPC A61B 5/150305; A61B 2050/005; A61B 5/150343; A61B 5/150389; A61B 5/150503; A61B 5/153; A61B 17/132; A61B 5/150328; A61F 13/00072; A61B 50/3001; A61F 13/36; A61B 5/150786; A61B 50/31; A61F 2013/00089; A61B 2050/314; A61B 5/15003

(57) ABSTRACT

Described herein are various systems, methods, apparatuses, and kits for phlebotomy. In certain embodiments, the phlebotomy kit or system includes a blood collection tube, a needle assembly, a holder for the blood collection tube, a bandage, a tourniquet, a wipe, and an absorbent pad. The disclosed systems, methods, apparatuses, and kits are useful for collecting a single small volume of blood, and collecting of a small volume of blood at remote non-hospital environments.

### RELATED APPLICATIONS

This application claims the benefit of priority to U.S. Provisional Patent Application Ser. No. 62/420731, filed Nov. 11, 2016, the contents of which are incorporated herein by reference in their entirety.

### TECHNICAL FIELD

The invention relates generally to phlebotomy kits. More particularly, the invention relates to portable kits and systems for collecting a small volume blood sample.

### BACKGROUND

Blood sample collection from a patient involves incision of a blood vessel by a needle and blood drawn into a collection vessel. This process (i.e., phlebotomy) is performed by phlebotomists, doctors, nurses, medical laboratory scientists and the like. To obtain blood from a patient, hypodermic needles, syringes, etc. are typically required.

### SUMMARY

Described herein are various systems, methods, apparatuses, and kits for phlebotomy.

Embodiments of the present invention described herein provide convenient single-use kits for blood collection that are inexpensive, readily disposable, portable, mobile, light, compact, and stackable. Embodiments of the present invention allow one to safely perform small sample blood collection (e.g., from 0.5 mL to 10 mL), not only in hospitals, but also in lesser equipped environments. Embodiments of the present invention conveniently include components for blood collection in a single small and portable kit, thereby promoting efficient blood collection processes for many purposes.

In one aspect, the present invention is directed to a single-use, portable kit for collecting a single small volume (e.g., from 0.5 to 10 mL, e.g., from 1 to 5 mL) of blood, which comprises (a) a blood collection tube comprising a pierceable septum over an open end of the blood collection tube, (b) a needle assembly comprising a first tip for intravenous puncture, and a second tip, for insertion into the blood collection tube through the pierceable septum, (c) a holder for the blood collection tube, (d) a bandage, (e) a tourniquet, (f) a wipe, and (g) an absorbent pad. In certain embodiments, the absorbent pad wraps the blood collection tube, the needle assembly, the holder, the bandage, the tourniquet, and the wipe. In certain embodiments, a height of the kit ranges from 5 to 7 inches, a width of the kit ranges from 5 to 7 inches, and a thickness of the kit ranges from 0.5 to 1 inch (e.g., wherein the kit contains and the absorbent pad wraps only one needle assembly).

In certain embodiments, the kit further comprises a sponge wrapped inside the absorbent pad. In certain embodiments, the kit further comprise a sealable bag, containing the blood collection tube, the needle assembly, the holder, the bandage, the tourniquet, the wipe, and the absorbent pad.

In certain embodiments, the absorbent pad is rectangular, having a first length in a first direction and a second length in a second direction. In certain embodiments, the first length is at least three times longer than the width of the kit and/or the sealable bag (e.g., wherein the first length is at least three times longer than the width of the kit; e.g., wherein the first length is at least three times longer than the width of the sealable bag) (e.g., the absorbent pad is folded four times in the first direction to be contained in the sealable bag). In certain embodiments, the second length is at least two times longer than the height of the kit and/or the sealable bag (e.g., wherein the second length is at least two times longer than the height of the kit; e.g., wherein the second length is at least two times longer than the height of the sealable bag) (e.g., the absorbent pad is folded twice in the second direction to be contained in the sealable bag).

In certain embodiments, the kit further comprises an identification tag. In certain embodiments, the identification tag is located on the blood collection tube. In certain embodiments, the identification tag has a unique barcode.

In certain embodiments, the blood collection tube has a volume of up to about 4 ml.

In another aspect, the present invention is directed to a system for remote non-hospital collection of a small volume (e.g., from 0.5 to 10 mL, e.g., from 1 to 5 mL) of blood, which comprises (a) a blood collection tube comprising a pierceable septum over an open end of the blood collection tube, (b) a needle assembly, (c) a holder, (d) a bandage, (e) a tourniquet, (f) a wipe, and (g) an absorbent pad. In certain embodiments, the absorbent pad wraps the blood collection tube, the needle assembly, the holder, the bandage, the tourniquet, and the wipe. In certain embodiments, the needle assembly comprises a first tip to access (e.g., puncture) a blood vessel. In certain embodiments, the needle assembly comprises a second tip to direct blood from the blood vessel to the blood collection tube through the pierceable septum. In certain embodiments, the holder comprises an opening to accommodate the blood collection tube, and a mounting aperture to connect to the second tip. In certain embodiments, the bandage protects a site of the blood collection. In certain embodiments, the tourniquet is elastic, so that, when the tourniquet is applied to a subject (e.g., upper arm), the tourniquet temporarily increases pressure in the blood vessel. In certain embodiments, the wipe comprises a sterilizing solution to sanitize the site of blood collection (e.g., upper arm). In certain embodiments, the absorbent pad provides protection (e.g., protection from mechanical damage during transport) to the system, as well as a clean field for working surface. In certain embodiments, a height of the kit ranges from 5 to 7 inches, a width of the kit ranges from 5 to 7 inches, and a thickness of the kit ranges from 0.5 to 1 inch.

In certain embodiments, the system further comprises a sponge wrapped inside the absorbent pad.

In certain embodiments, the system further comprises a sealable bag containing the blood collection tube, the needle assembly, the holder, the bandage, the tourniquet, the wipe, and the absorbent pad, wherein the sealable bag provides protection (e.g., protection from water, dust, contaminants) to the system.

In certain embodiments, the absorbent pad is rectangular, having a first length in a first direction and a second length in a second direction. In certain embodiments, the first length is at least three times longer than the width of the kit (e.g. the absorbent pad is folded four times in the first direction to be contained in the sealable bag). In certain embodiments, the second length is at least two times longer than the height of the kit (e.g., the absorbent pad is folded twice in the second direction to be contained in the sealable bag).

In certain embodiments, the system comprises an identification tag having a unique barcode. In certain embodiments, the identification tag is located on the blood collection tube.

In certain embodiments, the identification tag has a unique barcode.

In certain embodiments, the blood collection tube has a volume of up to about 4 ml.

In certain embodiments, the needle assembly further comprises a tubing connecting the first tip and the second tip.

In certain embodiments, the sealable bag facilitates transport of the system to a phlebotomist or a subject.

In another aspect, the present invention is directed to a system for collecting a small volume (e.g., 0.5, 1, 2, 3, or 4 mL) of blood, which comprises (a) a blood collection tube comprising a pierceable septum over an open end of the blood collection tube, (b) a needle assembly, (c) a holder, (d) a bandage, (e) a tourniquet, (f) a wipe, and (g) an absorbent pad. In certain embodiments, the needle assembly comprises a first tip to access (e.g., puncture) a blood vessel. In certain embodiments, the needle assembly comprises a second tip to direct blood from the blood vessel to the blood collection tube through the pierceable septum. In certain embodiments, the holder comprises an opening to accommodate the blood collection tube, and a mounting aperture to connect to the second tip. In certain embodiments, the bandage protects a site of the blood collection. In certain embodiments, the tourniquet is elastic, so that, when the tourniquet is applied to a subject (e.g., upper arm), the tourniquet temporarily increases pressure in the blood vessel. In certain embodiments, the wipe comprises a sterilizing solution to sanitize a site of blood collection (e.g., upper arm). In certain embodiments, the absorbent pad wraps the blood collection tube, the needle assembly, the holder, the bandage, the tourniquet, and the wipe, such that, when the absorbent pad is unwrapped, the blood collection tube, the needle assembly, the holder, the bandage, the tourniquet, and the wipe are positioned on the absorbent pad (e.g., on an absorbing layer of the absorbent pad). In certain embodiments, a height of the kit ranges from 5 to 7 inches, a width of the kit ranges from 5 to 7 inches, and a thickness of the kit ranges from 0.5 to 1 inch.

### Definitions

In order for the present disclosure to be more readily understood, certain terms are first defined below. Additional definitions for the following terms and other terms are set forth throughout the specification.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

As used herein, the term “designed” refers to an agent (i) whose structure is or was selected by the hand of man; (ii) that is produced by a process requiring the hand of man; and/or (iii) that is distinct from natural substances and other known agents.

As used herein, the term “subject” refers to any organism from which a biological sample is or may be obtained, e.g., for experimental, diagnostic, prophylactic, cosmetic, and/or therapeutic purposes. Typical subjects include animals (e.g., mammals such as mice, rats, rabbits, non-human primates, and/or humans). In certain embodiments, a subject is a human. In certain embodiments, a subject is suffering from or susceptible to one or more disorders or conditions. In certain embodiments, a subject displays one or more symptoms of a disorder or condition. In certain embodiments, subject is receiving or has received certain therapy to diagnose and/or to treat a disease, disorder, or condition.

As used herein, the term “substantially” refers to the qualitative condition of exhibiting total or near-total extent or degree of a characteristic or property of interest.

Unless defined otherwise, technical and scientific terms have the same meaning as commonly understood by one of ordinary skill in the art to which this invention belongs.

All references cited herein, including patent applications and publications, are incorporated by reference in their entirety.

### BRIEF DESCRIPTION OF THE DRAWINGS

The Drawings, which are comprised of at least the following Figures, is for illustration purposes only, not for limitation.

FIG. 1A shows an exemplary phlebotomy kit and depicts phlebotomy components included in the exemplary phlebotomy kit, according to an illustrative embodiment.

FIG. 1B shows an exemplary phlebotomy kit with phlebotomy components positioned on an absorbent pad after unwrapping the absorbent pad, according to an illustrative embodiment.

FIG. 1C shows an exemplary phlebotomy kit, illustrating how the exemplary phlebotomy kit can be contained in a sealable bag, according to an illustrative embodiment.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

The following description is for illustration and exemplification of the invention only, and is not intended to limit the invention to the specific embodiments described.

Unless defined otherwise, technical and scientific terms have the same meaning as commonly understood by one of ordinary skill in the art to which this invention belongs.

All references cited herein, including patent applications and publications, are incorporated by reference in their entirety.

It is contemplated that systems, devices, methods, and processes of the claimed invention encompass variations and adaptations developed using information from the embodiments described herein. Adaptation and/or modification of the systems, devices, methods, and processes described herein may be performed, as contemplated by this description.

Throughout the description, where articles, devices, and systems are described as having, including, or comprising specific components, or where processes and methods are described as having, including, or comprising specific steps, it is contemplated that, additionally, there are articles, devices, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

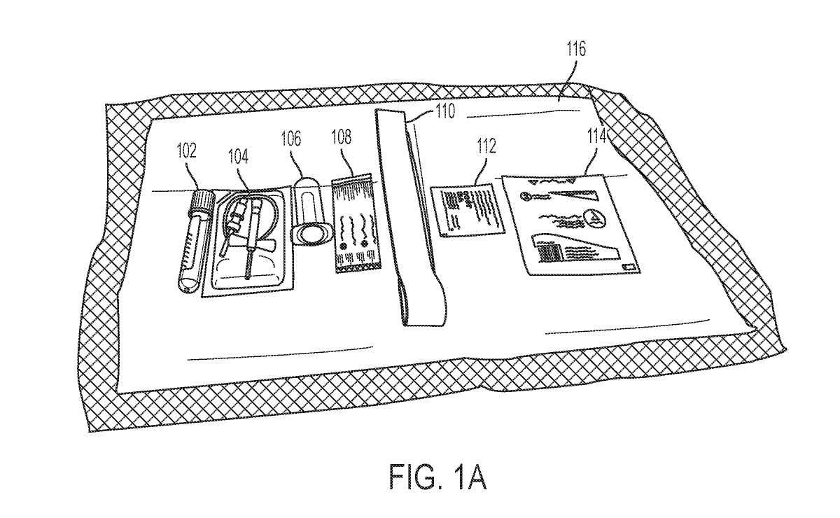

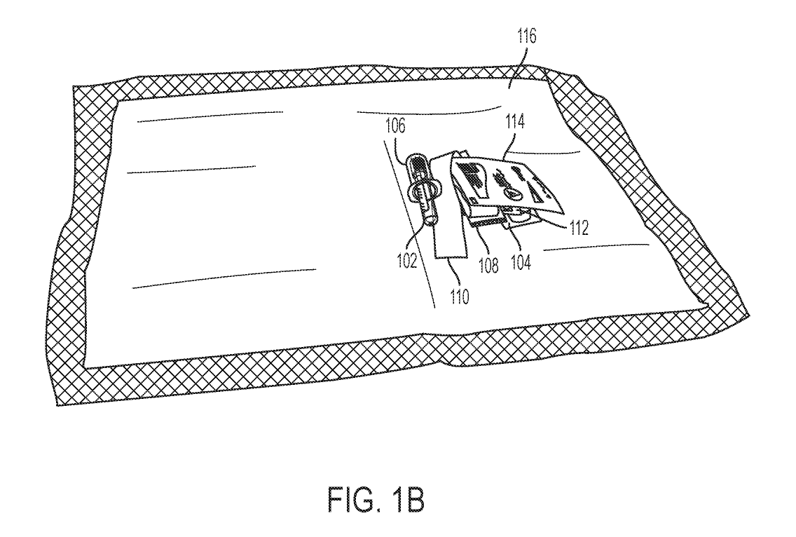

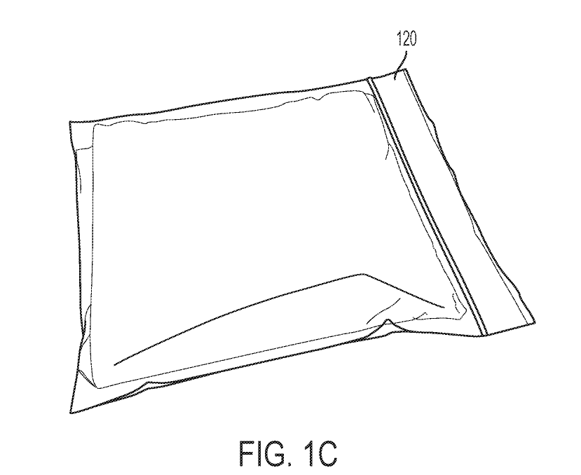

In certain embodiments, a kit for blood collection is light, compact, and/or stackable. The kit can be transported easily to a phlebotomist. The kit comprises components for phlebotomy, for example, blood collection tube 102, needle assembly 104, holder 106, bandage 108, tourniquet 110, wipe 112, sponge 114, and absorbent pad 116 (FIG. 1A). In certain embodiments, the kit (e.g., phlebotomy components) is contained in a sealable bag 120 (FIG. 1C). The sealable bag 120 may define the size of the kit. In certain embodiments, the sealable bag 120 provides protection from water, dust, contaminants, etc. In certain embodiments, the absorbent pad 116 wraps the phlebotomy components (e.g., blood collection tube 102, needle assembly 104, holder 106, bandage 108, tourniquet 110, wipe 112, sponge 114). The absorbent pad 116 is sized to wrap the components properly. For example, a rectangular absorbent pad 116 may have a first length in a first direction, which is at least three, four or five times longer than the width of the kit (or a sealable bag) (e.g., the absorbent pad 116 is folded four, five, or six times in the first direction to be contained in the sealable bag). The rectangular absorbent pad 116 may also have a second length in a second direction, which is at least three, four, or five times longer than the height of the kit (or the sealable bag) (e.g., the absorbent pad 116 is folded two times, three times, or four times in the second direction to be contained in the sealable bag). In certain embodiments, the absorbent pad 116 provides protection from mechanical damage (e.g., during transportation) to the kit. In certain embodiments, the absorbent pad 116 wraps the phlebotomy components such that, when the absorbent pad 116 is unwrapped, the phlebotomy components are positioned on the absorbent pad 116 (FIG. 1B). The phlebotomy components may be located in the center of the absorbent pad 116 after unwrapping. The phlebotomy components may be located on an absorbent layer of the absorbent pad 116 (e.g., not on an impervious layer).

### Blood Collection Tube

Any type of blood collection tube 102 may be used in accordance with the invention. In certain embodiments, a blood collection tube 102 has an open end, a closed end and a cylindrical wall therebetween defining a tube interior for accommodating blood. The blood collection tube 102 has an accessible closure (e.g., pierceable septum). The pierceable septum of the blood collection tube 102 may be constructed of a pierceable, self-sealing material such as an elastomeric material. The pierceable septum is capable of being punctured or pierced with a needle.

In certain embodiments, the pressure in the blood collection tube 102 is lower than the pressure in a blood vessel, so that the pressure difference causes blood to flow into the blood collection tube 102 without application of mechanical force.

In certain embodiments, the blood collection tube 102 has a volume a volume of up to about 1, 2, 3, 4, 5, 6, 7, 8, 9, or 10 ml.

### Needle Assembly

Any type of needle assembly 104 may be used in accordance with the invention. In certain embodiments, a needle assembly 104 includes a first tip for intravenous puncture and a second tip for insertion into the blood collection tube. The first tip and the second tip are interconnected through a tubing.

In certain embodiments, needles in the needle assembly 104 have a gauge within the range of about 25 to about 35.

### Holder

In certain embodiments, a holder 106 for a blood collection tube 102 includes a proximal end, a distal end and a tubular side wall extending between the ends. The proximal end of the holder 106 is widely open and defines an entrance to a tube receptacle within the tubular side wall. The distal end wall extends partly across the distal end of the holder 106 and has a mounting aperture that communicates with the tube receptacle. The mounting aperture in the distal end wall may include internal threads, movable jaws or similar structure for releasably receiving a needle assembly.

The holder 106 is generally available in various sizes (e.g., accommodating various sizes of blood collection tubes) and configurations. Holders can be any material of construction (e.g., polyethylene, glass, etc.)

The blood collection tube, needle assembly 104 and holder 106 are employed by first removing the packaging cover and connecting the second tip of the needle assembly 104 in the mounting aperture in the distal end wall of the holder.

### Bandage

Any type of suitably sized bandage 108 may be used in accordance with the invention. In certain embodiments, a bandage 108 has a first layer comprising a gauze pad, and a second layer comprising flexible material. The second layer may be adhesive and be adhered to skin. In certain embodiments, the bandage 108 is sterilized.

In certain embodiments, the bandage 108 is applied to a site of the blood collection in order to protect the site after the blood collection.

### Tourniquet

Any type of suitably sized tourniquet 110 may be used in accordance with the invention. A tourniquet 110 is used to control blood circulation by applying pressure upon the skin and underlying tissues of an arm. This pressure can be transferred to the walls of blood vessels. In certain embodiments, a tourniquet 110 is applied before puncturing a blood vessel with a needle. The tourniquet 110 may be applied about 4-5 finger widths above the site for phlebotomy.

Tourniquets according to the disclosed invention can include any type of tourniquet. In certain embodiments, a tourniquet 110 is a disposable. The tourniquet 110 may be non-latex. The tourniquet 110 comprises an elongated strip of material. The tourniquet 110 may be of various sizes, e.g., about ¾″ to about 1½″ wide and about 12″ to about 30″ in length. The thickness of the individual tourniquet 110 may be about 0.006 inches to about 0.024 inches. In certain embodiments, the tourniquet 110 has a modulus of about 1000 to about 3000 PSI, a tensile strength of about 1000 to about 3000 PSI.

### Wipe

In certain embodiments, the wipe 112 comprises a sterilizing solution. The sterilizing solution may be selected from the group consisting of chlorhexidine, iodophors, alcohol (e.g., ethyl alcohol, isopropyl alcohol), and combinations thereof. A phlebotomist or subject may sanitize the skin to be punctured for blood collection with the wipe.

### Sponge

Any type of suitably sized sponge 114 may be used in accordance with the invention. In certain embodiments, a sponge 114 is surgical gauze. In certain embodiments, the sponge 114 is applied to a site of the blood collection after removing the needle assembly 104 from the blood vessel in order to stop bleeding. Extra-pressure may be applied to the site of the blood collection in order to stop bleeding quickly.

### Absorbent Pad

Any type of suitably sized absorbent pad 116 may be used in accordance with the invention. In certain embodiments, the absorbent pad 116 absorbs spills or drops of liquid (e.g., blood) before, during or after phlebotomy. The absorbent pad 116 may comprise two layers with absorbent material located between the two layers. The top layer may be a non-woven pad and the bottom layer comprises an impervious film to enclose liquid spills. Inside the two layers is an absorbent media fabricated from tissue paper or wood pulp materials with a super absorbent polymer. In certain embodiments, the absorbent pad 116 is a flat sheet.

In certain embodiments, the absorbent pad 116 is rectangular. The absorbent pad 116 has a first length in a first direction and a second length in a second direction. The first length of the absorbent pad 116 may be about 10 to about 50 inches, 10 to about 40 inches, 20 to about 30 inches, or 20 to about 30 inches. The second length of the absorbent pad 116 may be about 10 to about 30 inches, or about 15 to about 20 inches.

### Sealable Bag

Any type of suitably sized sealable bag 120 may be used in accordance with the invention. In certain embodiments, a sealable bag 120 has an interior surface, exterior surface, and an opening large enough to accommodate the phlebotomy components. The bag opening may be sealed by a zip type fastener, a hook and loop fastener, slider, tap, top stop, pull tab, heat or the like.

### Use

In an exemplary method, a blood collection process includes the following steps, not necessarily in the order enumerated below:
1. Unwrap the absorbent pad.
2. Place the absorbent pad 116 under the site of the blood collection.
3. Apply the tourniquet 110 4-6 inches above the site of blood collection. Tighten the tourniquet.
4. Connect the second tip of the needle assembly 104 to the mounting aperture of the holder.
5. Cleanse the site of blood collection using the wipe. Let the site dry.
6. Hold skin with nondominant hand. Puncture the blood vessel with the first tip of the needle assembly 104 pointed up at about a 30 degree angle.
7. Lower the first tip toward skin after the first tip has entered the blood vessel.
8. Thread the needle along the path of the blood vessel.
9. Once the needle is in the blood vessel and blood flows out of the blood vessel, insert the blood collection tube 102 into the holder.
10. Press the blood collection tube 102 into the second tip of the needle assembly, so that it pierces the septum.
11. Collect the blood.
12. Release the tourniquet.
13. Remove the needle assembly 104 from the blood vessel.
14. Press the site of the blood collection firmly with the sponge 114 until bleeding stops.
15. Apply the bandage 108 on the site of the blood collection.

Elements of different implementations described herein may be combined to form other implementations not specifically set forth above. In addition, the logic flows depicted in the figures do not require the particular order shown, or sequential order, to achieve desirable results. Various separate elements may be combined into one or more individual elements to perform the functions described herein. In view of the structure, functions and apparatus of the systems and methods described here, in some implementations.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A single-use, portable kit for collecting a single small volume of blood, the kit comprising:
(a) a blood collection tube comprising a pierceable septum over an open end of the blood collection tube;
(b) a needle assembly comprising a first tip for intravenous puncture, and a second tip, for insertion into the blood collection tube through the pierceable septum;
(c) a holder for the blood collection tube;
(d) a bandage;
(e) a tourniquet;
(f) a wipe; and
(g) an absorbent pad, wrapping the blood collection tube, the needle assembly, the holder, the bandage, the tourniquet, and the wipe;
wherein the single small volume of blood is from 0.5 to 10 mL,
wherein a height of the kit ranges from 5 to 7 inches,
wherein a width of the kit ranges from 5 to 7 inches, and
wherein a thickness of the kit ranges from 0.5 to 1 inch.

2. The kit of claim 1, further comprising a sponge wrapped inside the absorbent pad.

3. The kit of claim 1, further comprising a sealable bag, containing the blood collection tube, the needle assembly, the holder, the bandage, the tourniquet, the wipe, and the absorbent pad.

4. The kit of claim 3, wherein the absorbent pad is rectangular, having a first length in a first direction and a second length in a second direction, and wherein the first length is at least three times longer than the width of the kit and/or the sealable bag.

5. The kit of claim 4, wherein the second length is at least two times longer than the height of the kit and/or the sealable bag.

6. The kit of claim 1, further comprising an identification tag.

7. The kit of claim 6, wherein the identification tag is located on the blood collection tube.

8. The kit of claim 6, wherein the identification tag has a unique barcode.

9. The kit of claim 1, wherein the blood collection tube has a volume of up to about 4 ml.

10. A system for remote non-hospital collection of a small volume of blood, the system comprising:
(a) a blood collection tube comprising a pierceable septum over an open end of the blood collection tube;
(b) a needle assembly;
(c) a holder;
(d) a bandage;
(e) a tourniquet;
(f) a wipe; and
(g) an absorbent pad, wrapping the blood collection tube, the needle assembly, the holder, the bandage, the tourniquet, and the wipe;
wherein the single small volume of blood is from 0.5 to 10 mL,
wherein the needle assembly comprises a first tip to access a blood vessel,
wherein the needle assembly comprises a second tip to direct blood from the blood vessel to the blood collection tube through the pierceable septum,
wherein the holder comprises an opening to accommodate the blood collection tube, and a mounting aperture to connect to the second tip,
wherein the bandage protects a site of the blood collection,
wherein the tourniquet is elastic, so that, when the tourniquet is applied to a subject, the tourniquet temporarily increases pressure in the blood vessel,
wherein the wipe comprises a sterilizing solution to sanitize the site of blood collection, wherein the absorbent pad provides protection to the system, as well as a clean field for working surface,
wherein a height of the kit ranges from 5 to 7 inches,
wherein a width of the kit ranges from 5 to 7 inches, and
wherein a thickness of the kit ranges from 0.5 to 1 inch.

11. The system of claim 10, further comprising a sponge wrapped inside the absorbent pad.

12. The system of claim 10, further comprising a sealable bag containing the blood collection tube, the needle assembly, the holder, the bandage, the tourniquet, the wipe, and the absorbent pad, wherein the sealable bag provides protection to the system.

13. The system of claim 12, wherein the absorbent pad is rectangular, having a first length in a first direction and a second length in a second direction, and the first length is at least three times longer than the width of the kit.

14. The system of claim 13, wherein the second length is at least two times longer than the height of the kit.

15. The system of claim 10, further comprising an identification tag.

16. The system of claim 15, wherein the identification tag is located on the blood collection tube.

17. The system of claim 15, wherein the identification tag has a unique barcode.

18. The system of claim 10, wherein the blood collection tube has a volume of up to about 4 ml.

19. The system of claim 10, wherein the needle assembly further comprises a tubing connecting the first tip and the second tip.

20. The system of claim 12, wherein the sealable bag facilitates transport of the system to a phlebotomist or a subject.

21. A system for remote non-hospital collecting a small volume of blood, the system comprising:
(a) a blood collection tube comprising a pierceable septum over an open end of the blood collection tube;
(b) a needle assembly;
(c) a holder;
(d) a bandage;
(e) a tourniquet;
(f) a wipe; and
(g) an absorbent pad;
wherein the single small volume of blood is from 0.5 to 10 mL,
wherein the needle assembly comprises a first tip to access a blood vessel,
wherein the needle assembly comprises a second tip to direct blood from the blood vessel to the blood collection tube through the pierceable septum,
wherein the holder comprises an opening to accommodate the blood collection tube, and a mounting aperture to connect to the second tip,
wherein the bandage protects a site of the blood collection,
wherein the tourniquet is elastic, so that, when the tourniquet is applied to a subject, the tourniquet temporarily increases pressure in the blood vessel,
wherein the wipe comprises a sterilizing solution to sanitize a site of blood collection,
wherein the absorbent pad wraps the blood collection tube, the needle assembly, the holder, the bandage, the tourniquet, and the wipe such that, when the absorbent pad is unwrapped, the blood collection tube, the needle assembly, the holder, the bandage, the tourniquet, and the wipe are positioned on the absorbent pad,
wherein a height of the kit ranges from 5 to 7 inches,
wherein a width of the kit ranges from 5 to 7 inches, and
wherein a thickness of the kit ranges from 0.5 to 1 inch.
